(19) United States
(12) Patent Application Publication
(10) Pub. No.: US 2007/0071684 A1
(43) Pub. Date: Mar. 29, 2007

(54) Process for the preparation of surface coatings and films

(76) Inventors: Nigel Stuart Crutchley, Leeds (GB); James Thomas Guthrie, Leeds (GB); Derek Alfred Wheeler, Surrey (GB); Stephen John Lenon, Surrey (GB)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 10/573,380

(22) PCT Filed: Sep. 23, 2004
(86) PCT No.: PCT/GB04/04064

§ 371 Date: Mar. 24, 2006

(30) Foreign Application Priority Data
Sep. 25, 2003 (GB) 0322485.4

(51) Int. Cl. A61K 9/28

(52) U.S. Cl. 424/9.52; 427/2.14

(57) ABSTRACT

A method of coating the surface of a substrate which comprises the steps of: i) contacting the surface with a polymerisable mixture comprising one or more polymerisable components and containing suspended droplets of a biliquid foam or of a high internal oil phase emulsion, the said droplets being stabilised by a non-reactive surfactant; and ii) polymerising the coating to form a polymer comprising the droplets entrapped therein.

The present invention relates to a process for the preparation of surface coatings or films, for example dry films, in which one or more oils or oil-soluble substances are encapsulated as discrete oil droplets within the surface coating or film.

The entrapment of oils or oil soluble substances (especially perfumes and coloured dye precursors) in microcapsules and their subsequent coating onto paper and other surfaces is well known in the art. Microcapsules of this type comprise individual droplets of oil or oil soluble substances (of size ranging from sub-micrometre to tens of millimetres in diameter) around which polymer walls have Usually such microcapsules are prepared as an aqueous suspension which is then capable, with the addition of suitable modifying reagents, of being sprayed or printed onto paper and other surfaces. The object in so doing is usually to prevent the evaporation of volatile substances (for example, perfumes) or the degradation or chemical reaction of oil soluble species (for example, colourless dye precursors) until the microcapsules are broken by the application of shear forces by scratching or scraping the coated surface with the consequent release of their contents. Such coatings find major uses, for example, in the forms of “scratch and sniff” perfume coatings or NCR (No Carbon Required) paper.

However, such coatings and the use of microcapsules that form them suffer from a number of disadvantages.

Firstly, the process by which microcapsules are formed is a lengthy and uncertain one in which control over temperature, pH and the absence of any form of contamination is essential. The formation of microcapsules, for example, by complex coacervation from gelatine and an anionic complexing species such as gum acacia takes many hours and demands very close control of pH, temperature and cooling rate. Similarly, the formation of microcapsule walls from aminoplast resins, such as melamine formaldehyde or urea-formaldehyde, takes at least eight hours, during which precise control over all controllable parameters needs to be effected. Moreover, the effectiveness and completeness of any individual encapsulation process, and therefore the quality of the microcapsules so formed, depends largely upon the chemical nature of the oil and/or oil soluble substances being encapsulated.

A further disadvantage of microencapsulation is that the thickness and therefore the strength of the microcapsule wall is variable and is not easily controllable and varies with the nature of the oil or oil-soluble substances being encapsulated. Thus, microcapsules made by the same process, but from different oils, may have widely differing strengths and resistance to breakage during the printing process and during subsequent storage and use.

A yet further disadvantage of microencapsulation is the limited number of chemical processes and the limited number and type of polymeric wall materials that are available to form them. The choice as to the properties of the wall materials is consequently limited with regard to their flexibility, tensile strength, permeability, chemical inertness, mammalian toxicity and other properties including solubility and melting point (if any). In addition, some of the chemicals commonly used in the wall forming process are themselves highly irritating and may themselves be toxic. An example of such toxicity is seen in the use or release of formaldehyde (a potential carcinogen) during the manufacture of aminoplast resin walls. Moreover, the remaining traces of formalin in the resulting microcapsule suspension are virtually impossible to eliminate to below the required levels for uses of microcapsules and requires special precautions to be taken during the manufacturing process.

A further disadvantage of microcapsules which are used in surface coatings is that the microcapsule walls have a limited deformability. Thus, they can only be deformed to a limited extent during the surface coating process (typically a printing process) before they will rupture and prematurely release their contents. The extent of their ability to deform when squeezed, for example, between nip rollers on a printing press set with a gap smaller than the average diameter of the microcapsules, depends partly upon the tensile properties of the polymer wall, its thickness and on the size of the microcapsules being squeezed.

Other methods for coating paper and other surfaces with mobile oils are known, but these are generally inferior to coating with microcapsules since they do not effectively trap and protect the oils from evaporation or degradation during manufacture and during subsequent storage prior to use. For example, perfumes may be sprayed or otherwise coated on to paper surfaces in order to give paper products a pleasant smell—as for instance, with perfumed drawer liners wherein the coating is a sprayed-on perfume and not a sprayed-on microcapsuled perfume. Such products have a limited shelf life (because of the premature evaporation of the perfume) and the outer packaging of the product is usually the only (and relatively ineffective) barrier to the loss of perfume of other volatile substances during storage.

WO 02/051536 describes a process for the encapsulation of an emulsion in which a water-in-oil or an oil-in-water emulsion is prepared from a polymerisable emulsifier, at least one polyfunctional comonomer, at least one hydrophilic liquid and at least one hydrophobic liquid. The mixture is polymerised by means of UV curing and/or initiators. During which polymerisation the polymerisable emulsifier and the polyfunctional comonomer react together to form a matrix that entraps the emulsion in the microcapsules that have a particle size of from 70 nm to 5 μm.

WO 99/05229 describes a method of coating the surface of a substrate in which the surface is contacted with a dispersion of a pre-formed, film forming polymer, the dispersion containing droplets of a biliquid foam or of an emulsion, and allowing the dispersion to dry so as to coat the surface with a coating comprising the droplets trapped within a film of the polymer. This process suffers from the disadvantage that if the film forming polymer suspensions are aqueous, the drying of the dispersion requires a long period of time at room temperature or the application of heat. If the film forming polymer dispersions contain high levels of more volatile polar solvents, then appropriate measures are needed to prevent emissions into the environment in the drying process. Furthermore, since the polymer is pre-formed, evaporation of the solvent may result in significant shrinkage in the films. Additionally heating cannot be used to speed up drying and film formation if heat-sensitive oils are used.

We have now developed a process for the preparation of films or coatings, for example dry films, which encapsulate droplets of a biliquid foam or of a high internal phase oil-in-water emulsion therein. The process does not suffer from the disadvantages of the process of WO 99/05329. In the process of the present invention the fluid mixture turns into a solid polymer at the same time as encapsulating the oil droplets within the solid polymer system.

Accordingly, the present invention provides a method of coating the surface of a substrate which comprises the steps of:
i) contacting the surface with a polymerisable mixture comprising one or more polymerisable components and containing suspended droplets of a biliquid foam or of a high internal oil phase emulsion, the said droplets being stabilised by a non-reactive surfactant; and
ii) polymerising the coating to form a polymer, preferably a film of a polymer, comprising the droplets entrapped therein.

Using the method of the present invention, a surface coating is obtained which comprises a polymer or polymer film in which the droplets of the biliquid foam or a high internal oil phase emulsion are entrapped. These systems are preferred since they contain low levels of water.

Preferably a biliquid foam is used. Biliquid foams are known in the art in which small droplets of a predominantly non-polar liquid such as an oil are encapsulated in a surfactant-stabilized film of a hydrogen bonded liquid, such as water, and separated from one another by a thin film of the hydrogen bonded liquid. The water or other hydrogen bonded liquid thus forms the continuous phase in biliquid foam compositions.

Biliquid foams are disclosed in the following literature references by Sebba:

“Biliquid Foams”, J. Colloid and Interface Science, 40 (1972) 468-474; and “The Behaviour of Minute Oil Droplets Encapsulated in a Water Film”, Colloid Polymer Sciences, 257 (1979) 392-396.

The biliquid foam or high internal oil phase emulsion that is used in the present invention will generally comprise at least 70 percent by weight of the oil phase, preferably greater then 85 percent by weight and more preferably greater than 90 percent by weight of the oil phase. The external phase is polar and may consist of water or water in admixture with other polar solvents such as C₁₋₄ alcohols or organic oxygenates. The external phase may also comprise one or more polymerisable components, such as N-vinyl pyrrolidone.

The polymerisable mixture will generally comprise from 1 to 50 percent by weight of the biliquid foam or high internal oil phase emulsion, preferably from 20 to 40 percent by weight thereof.

The biliquid foam or high internal oil phase emulsion is stabilized in the present invention by a non-reactive surfactant. By the term “non-reactive surfactant” as used herein we mean a surfactant that does not polymerise with, or react with, the polymerisable components of the polymerisable mixture. Accordingly, on polymerising the polymerisable mixture, the formation of discrete microcapsulates will be avoided. If a polymerisable surfactant is used, there may arise a problem of a shell forming around the discrete droplets, forming microcapsules. The crosslinking at the droplet interface could limit the diffusion of the oil, such as a fragrance or aroma, from the droplet into the polymer films or coating and then into the environment, so that faster controlled release could riot be achieved.

In carrying out the process of the present invention the polymerisable components within the coating are polymerized to form a polymer or polymer film within which the oil-containing droplets are entrapped.

Thicker films or coatings can be polymerised in the presence of certain oils that do not absorb radiation, such as mineral oils. The oil can give deeper penetration of the radiation than could be achieved if the oil droplets were not present.

Polymerisation is generally defined as the formation of a polymer chain by the linking of repetitive monomer or oligomer subunits. Monomers are low molecular weight components for example which have a degree of unsaturation (carbon double bonds). They may be mono- or polyunsaturated. Oligomers (or pre-polymers) are larger molecular entities and are usually bifunctional, for example having two double bonds. The final characteristics of the polymer can be manipulated by blending monomers/oligomers of different chemical nature and varying degrees of unsaturation, for example to ensure that the final systems characteristics match or are consistent with the final application.

There are three major types of polymerisation, namely, free radical, cationic and anionic polymerisation.

Free radical polymerisation relies upon the generation of radical species that have unpaired electrons and are highly reactive. The formation of these highly excited radical states requires the input of additional energy from an outside source. Electron beam radiation causes the formation of radical species directly within the system by bombarding the monomers with electrons to disrupt the double bonds causing the formation of the radicals. The electron beam process is however energy intensive and also has the disadvantage of being limited to surface curing (1 to 2 microns in thickness) and limited largely to clear-coats. An alternative strategy is usually often adopted. This involves the incorporation of a photoinitiator into the formulations. Thus UV-curing processing becomes a highly attractive option. UV-curing relies on the presence of a suitable photoinitiator. A photoinitiator is a molecule that strongly absorbs light energy usually in the UV spectral region causing it to self-cleave (unimolecular scission). Other initiator systems involve the complexing of the photoinitiator with an hydrogen-atom donor (for example, a tertiary amine). The resulting input of UV energy causes the excitation of the complex (exiplex), resulting in the formation of the required radical species. This is a bimolecular process.

Photo-cationic polymerisation differs from free radical polymerisation with respect to the types of monomers, initiators and viscosity modifiers used. Instead of a free radical being the reactive species the photoinitiator releases a strong Lewis acid or Bronsted acid. These acids subsequently initiate the cationic polymerisation.

Photo-anionic polymerisation is also different from free radical polymerisation. Here the reactive species is a base that initiates the polymerisation.

Monomers/oligomers which may be used in free radical polymerization process will generally contain one or more vinyl and/or acrylic groups. Suitable vinyl monomers include, for example, monoalkenyl arene monomers such as styrene α-methylacrylate, chloromethylstyrene, vinylethylbenzene and vinyl toluene; acrylate or methacrylate esters such as 2-ethylhexyl(meth)acrylate, n-butyl(meth)acrylate, isobutyl(meth)acrylate, t-butyl (meth)acrylate, hexyl(meth)acrylate, n-butyl (meth)acrylate, lauryl(meth)acrylate, isodecyl (meth)acrylate, propoxylated neopentyl glycol di(meth)acrylate, melamine pent (meth)acrylate, polyethylene glycol di(meth)acrylate, trimethylpropane di(meth)acrylate, 1,6-hexanediol di(meth)acrylate, bisphenol A ethoxylate di(meth)acrylate, 2-phenoxyethyl(meth)acrylate, tripropylene glycol di(meth)acrylate, ethoxylated or propoxylated trimethylolpropane tri(meth)acrylate, dipropylene glycol di(meth)acrylate, diethylene glycol di(meth)acrylate, tetrahydrofurfuryl(meth)acrylate, n-vinyl caprolactam or N-vinyl pyrrolidone, dipentaerythritol monohydroxy pent(meth)acrylate, dipentaerythritol hex(meth)acrylate, conjugated diolefins such as butadiene, isoprene, and piperylene, allenes such as allene, methyl allene and chloroallene; olefin halides such as vinyl chloride, vinyl fluoride and polyfluoroolefins; unsaturated polyesters; vinyl siloxanes; acrylated siloxanes; vinyl ether/unsaturated ester systems such as maleates, fumarates or citraconates; N-vinylformamide; N-(n-hexyl)maleimide; N-arylimides; N-allyl imides and N-arylmaleimides, or mixtures thereof. Oligomers that may be used in the free radical polymerisation process include aliphatic urethane (meth)acrylate oligomers and aliphatic polyester/ether urethane(meth)acrylate mixtures as well as polyester (meth)acrylates, epoxy(meth)acrylates and phenolic (meth)acrylates.

Monomers/oligomers that may be used in cationic polymerisation processes include epoxides, such as cycloaliphatic epoxides; glycidyl ethers; diglycidyl ethers; propenyl ethers; 2- and 4-alkoxy styrene and polyfunctional vinyl ethers such as aliphatic polyurethane vinyl ethers, polyethyl divinyl ethers and aromatic polyurethane vinyl ethers, or mixtures thereof. Typical initiators for these systems are often referred to as latent acid initiators. These include diazonium salts; diarylidonnium and triaryl sulphonium salts; pyrillium, thiopyrillium and N-alkoxypyridinium compounds, all bearing non-nucleophilic counterions such as tetrafluoroborate, hexafluorophosphate, hexafluoroarsenate and hexafluoroantimonate. Sensitisers may be included in addition to these salts to extend the spectral sensitivity of onium salts. These sensitisers include anthracene, perylene, phenothiazine, Michler's ketone, xanthone, thioxanthone, benzophenone and acetophenone. The sensitisers may be in water-soluble and/or oil-soluble and/or organic solvent soluble form.

Monomers/oligomers that may be used in anionic polymerisation processes include epoxides, multifunctional (meth)acrylates and alpha-cyano(meth)acrylates. Examples also include the Michael addition reaction of a malonate polyester with a multifunctional (meth)acrylate.

Monomers with active groups such as fungicides, adhesion-promoters, flame-retardants, biocides or other functionality, for example to protect or assist the integrity of the films or coating and/or to enhance the application, may also be used.

Initiators for the anionic polymerisation reaction include tertiary amine salts of alpha-ketocarboxylic acids, suitably substituted carbamates(urethanes), phenylammonium n-butyltriphenyl borate salts, ferrocenes and metal amine salts.

The preferred approach in this invention is the use of radical, especially free-radical, polymerisation. Cationic initiation may be very slow, especially in the presence of moisture, whilst anionic initiation suffers from the lack of commercial initiator systems and the sensitivity to carbon dioxide from air.

Hybrid polymerization processes may also be used in mixtures of, for example, blocked isocyanates, acrylate diluents and epoxide prepolymers, vinyl ether diluents and acrylate prepolymers or acrylate diluents and vinyl ether prepolymers may be used.

Radical polymerisation initiators which may be used in the present invention may be water soluble and/or oil soluble and/or organic solvent soluble.

Water soluble initiators which may be used include, for example, potassium persulphate or sodium persulphate and various redox systems such as ammonium persulphate together with sodium metabisulphite. Other options include derivatized (water-solubilising groups) versions of the solvent/oil soluble photoinitiator molecules. Oil soluble initiators which may be used include, for example, azo compounds such as α,α-azo-bisbutyronitrile and peroxides such as benzoyl peroxide, methylethylketone peroxide, di-2-ethylhexylperoxydicarbonate and lauroyl peroxide. Other initiators which may be used include zinc carbonate, 1-hydroxy-cyclohexyl-phenylketone, 2-hydroxy-2-methyl-1-phenyl-1-propanone, 2-hydroxy-1-[4-(2-hydroxyethoxy)phenyl]2-methyl-1-propanone methylbenzoxyl formamate, 2-benzyl-2(dimethylamino)-1-[4-[4-morpholinyl)-phenyl]-1-butanone, 2-methyl-1-[4-(methylthio)phenyl]-2-(4-morpholinyl)phenyl-1-butanone, benzoin methyl ether, benzoin ethyl ether, α,α-diethoxyacetophenone, α,α-diethoxy-α-phenyl-acetophenone, 4,4′-bis(dimethyl-amino)benzophenone, ferrocene, xanthone, thioxanthone, 1-chloro-4-propoxy-thioxanthane, diphenyl(2,4,6-trimethylbenzoyl)-phosphine oxide, bis(eta 5-2,4-cyclopentadien-1-yl)bis[2,6-difluoro-3-(1H-pyrrol-1-yl)phenyl]titanium, iodonium(4-methyl-phenyl) [4-(2-methylpropyl)phenyl]-hexafluorophosphate-(1-),decabromodiphenyl oxide, pentachlorobenzene, pentabromomono-chloro cyclohexane, 2-ethyl anthraquinone, 1-(chloroethyl)-naphthalene, desyl chloride, chlorendic anhydride, naphthalene sulphonic chloride and 2-bromoethyl ethyl ester.

The initiator will generally be added to the monomer or oligomer. Combinations of initiators may also be used. The initiator may be present in an amount of from 0.005 to 20 percent by weight, preferably from 0.1 to 20 percent by weight, more preferably from 0.1 to 5 percent by weight of the composition and still more, preferably from 1 to 4 percent by weight of the composition.

The polymerisable mixture that is coated onto a substrate in the process of the present invention is, for example, polymerised by electron beam, UV radiation, visible radiation, near infra-red, thermal or gamma radiation. UV radiation is preferably used.

Further additives that may be used in the coating compositions include chain transfer agents, such as tertiary alcohol amines, triethanolamine, N-methyl diethanolamine, N,N-dimethyethanolamine or substituted morpholines such as N-methylmorpholine. Other additives that may be used are adhesion promoters, wetting agents, slip agents, preservatives, dyes, defoamers, inorganics (for example pigments, silicas, clays, etc.) photo-sensitisers, waxes such as solid or semi-solid waxes (can be used to prevent/reduce oxygen inhibition of polymerisation), unreactive preformed polymers and rheology modifying agents.

Coating may be carried out by any method, such as by printing, especially by screen-printing, gravure printing, flexographic printing, lithographic printing, ink jet printing or pad printing. Coating may also be carried out by, for example, spray-coating, roller coating, dip coating, blade, brush, pad or extrusion coating, including pen application using a writing implement.

The present invention has application in a wide variety of areas. An example of a suitable area is that of dental applications. Dental light-cure composites, often referred to as “white fillings” or “synthetic porcelain” are in the form of a paste or viscous liquid that can be manipulated and shaped and then polymerised with a special light, typically in a blue-wavelength visible spectrum. Such dental composites comprise synthetic resins, diluents, cross-linking diluents, initiators, additives and ceramic reinforcing filler particles. The filler particles may, for example, comprise finely ground quartz, borosilicate, lithium-aluminium-silicate glass and/or amorphous silica. Typical dental resin systems comprise the reaction product of bisphenol A and glycidyl methacrylate. Diluents include, for example, methyl methacrylate and cross-linking diluents include, for example, triethylene glycol dimethacrylate and ethylene glycol dimethacrylate. Typical initiators include benzoyl peroxide, 9,10-phenanthrene quinone, camphorquinone, benzil and N,N-diethyl aminoethyl methacrylate. In the method of the present invention such a formulation would additionally comprise a biliquid foam or high internal oil phase emulsion prior to polymerisation. When the system is exposed to the light radiation and polymerisation occurs, the droplets would be entrapped in the dental filling. The droplets may comprise, for example, an active material such as a fluoride, a drug, a flavour or a breath freshener, either neat or in solution, which may be released over time by diffusion to deliver the active material into the mouth.

The present invention includes within its scope a surface coating prepared according to the above described method in which droplets of a biliquid foam or a high internal oil phase emulsion are entrapped within a polymer such as a polymer coating or film.

The polymer or polymer film may be selected so that the entrapped oil phase is releasable from the coating upon the application of shear forces to the polymer or polymer film. In the instance where the substance is, or contains, a perfume, a “scratch and sniff” coating may be produced.

The polymer or polymer film may be selected so that the oil is releasable from the coating by the action of a chemical release agent on the polymer. The oil may be released at a determined pH, or by contact of the polymer or polymer film with water, or other predetermined solvent.

The polymer or polymer film may be selected so that the non-polar substance is released from the coating by the application of heat to the polymer.

The polymer or polymer film may be partially or wholly crosslinked.

It should be noted that all of these release mechanisms are difficult or impossible to achieve with prior art technology because of the limited choice of wall materials from which microcapsules can be made as noted above.

Methods and coatings in accordance with the invention will now be described with reference to the accompanying drawings in which:—

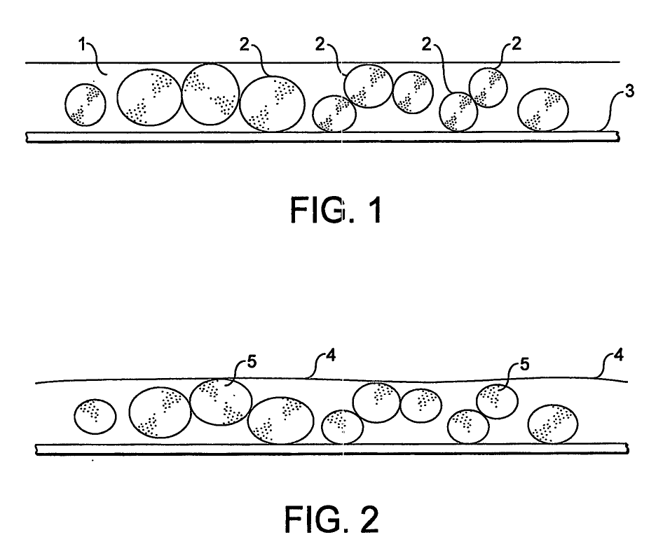

FIG. 1 shows a biliquid foam in a dispersion of a polymerisable mixture applied to a surface; and

FIG. 2 shows a surface coating after polymerisation of the polymerisable mixture of FIG. 1.

FIGS. 1 and 2 illustrate a method for coating the surface 3, such as a sheet, powder, film, fibre or mould including a cavity, of a substrate comprising the steps of:

contacting the surface 3 with a polymerisable mixture 1, the mixture 1 containing droplets 2 of a suspended biliquid foam or high internal oil phase emulsion; and

polymerising the mixture 1 so as to coat the surface with a coating comprising the droplets 5 trapped within a film 4 of said polymer.

The polymer film thus becomes a surface coating containing a plurality of suspended but intact oil droplets protected by the polymer film and depending for their integrity, strength, ease and method of rupture, chemical inertness and permeability on the structure, thickness and nature of the polymeric material.

It is preferable to use a biliquid foam, although the use of high internal oil phase emulsions is within the scope of the invention.

FIGS. 1 and 2 indicate diagrammatically the microscopic structure and appearance of a biliquid foam entrapped in a polymerisable mixture coated onto a surface before polymerisation (FIG. 1) and after polymerisation (FIG. 2). In FIG. 1, the polymerisable mixture 1 is coated onto a suitable surface 3. Droplets of biliquid foam 2 are trapped in the surface. These are typically 1 to 10 micrometres in diameter. FIG. 2 indicates the appearance of the polymerised film 4. The thickness of this film 4 will be similar to that of the uncured coating. The biliquid foam droplets 5 can be seen to be somewhat flattened (to an extent that is dependent upon the film thickness) but intact, with a surface covering of polymer film 4.

It is possible to make biliquid foams comprising mainly natural oils (for example, soya bean oil and sunflower oil), kerosenes, mineral oils, perfumes, essential oils, fragrances, aromas, organic solvents (for example, hexane, cyclohexane, chloroform, carbon tetrachloride and the like), silicone oils and their derivatives (such as dimethicones and cyciomethicones), fatty alcohols and their derivatives (for example isopropyl palmitate, isopropyl myristate) and most other non water-soluble liquids such as functional oils and non-polar liquids. The present invention may utilise any or all of the above singly or in combination or any other oil or non water-soluble, including non-polar, substances capable of existing in liquid form in the temperature range −50° C. to 200° C. but typically 3° C. to 90° C. and preferably 10° C. to 30° C. The oil phase may, for example, comprise other components such as solutions or neat actives or drugs.

The present invention provides a means of controlling the rate of release of the entrapped oil by exercising control over the concentration of the polymerisable component(s) in solution or suspension, and thereby controlling the thickness and strength of the polymer or polymer film deposited.

The present invention also includes within its scope a stand alone polymer or polymer film which is obtained by removing the surface coating prepared in the manner as hereinbefore described from the substrate on which it is formed.

The surface coating or film of the present invention or prepared by the process of the present invention can be used in many applications, for example in cosmetic, aesthetic, medical, dental or pest control applications. Examples of suitable applications are fragranced coatings, such as for use in automotive and room fresheners, insect repellents, fragranced coatings for packaging, greeting cards and stationary, security coatings including tamper proof coatings (for example comprising chemically reactive dyes, or thermo-chromic or photo-chromic dyes) or security inks, coatings for sun glasses and optics, dental fillings including actives such as drugs and flavours or architectural coatings including flame-retardant oils or oil compositions.

The present invention will be further described with reference to the following Examples.

### EXAMPLE 1

Preparation 1

A biliquid foam was prepared from the following ingredients.

| Ingredients | % |
|---|---|
| Aqueous phase | |
| Water | 9.895 |
| Sodium lauryl ether sulphate | 0.10 |
| Kathon CG | 0.005 |
| Oil phase | |
| Medium liquid white oil | 89.1 |
| Volpo V4 | 0.9 |
| | 100.00 |

The biliquid foam was prepared by adding the oil phase to the aqueous phase and stirring with a paddle stirrer at 200 rpm initially, increasing to 600 rpm.

Formulation 1

| Ingredients | Weight (g) | % |
|---|---|---|
| Preparation 1 | 0.55 | 18.77 |
| Ebecryl 2001(UCB) | 2.0 | 68.26 |
| Water | 0.28 | 9.95 |
| Darocur 4265(Ciba) | 0.10 | 3.42 |
| photoinitiator | | |
| Total | 3.93 | 100.00 |

The ingredients were mixed together, with preparation 1 being added last. The formulation was then applied as a 100 micrometre thick coating to a substrate using a calibrated slot film applicator. The sample was then cured, using a GEW bench UV curing system with power level of 100 W/cm and a conveyor speed of 0.1 m/second, with several passes under the UV lamp to ensure through cure.

### EXAMPLE 2

Preparation 2

A biliquid foam was prepared from the following ingredients.

| Ingredients | % |
|---|---|
| Aqueous phase | |
| Water | 9.895 |
| Sodium lauryl ether sulphate | 0.1 |
| Kathon CG | 0.005 |
| Oil phase | |
| Mineral oil | 89.1 |
| Volpo L3 | 0.9 |
| Total | 100.00 |

The biliquid foam was prepared by adding the oil phase to the aqueous phase and stirring using a paddle stirrer at an initial speed of 110 rpm rising to 525 rpm, until an average particle size of 9 μm was achieved.

Formulation 2

| Ingredients | Weight (g) | % |
|---|---|---|
| Preparation 2 | 0.27 | 20.45 |
| Aliphatic urethane diacrylate CN 981 | 0.668 | 50.61 |
| Cray Valley | | |
| Poly[oxy(methyl-1,2-ethanediyl)]Actilane | 0.332 | 25.15 |
| 421(Akzo Nobel) | | |
| Darocur 1173(Ciba) | 0.05 | 3.79 |
| photoinitiator | | |
| Total | 1.32 | 100.00 |

The ingredients were mixed together, with preparation 2 being added last. The formulation was then applied as a 100 micrometre thick coating to a substrate using a calibrated slot film applicator. The sample was then cured, using a GEW bench UV curing system with power level of 100 W/cm and a conveyor speed of 0.1 m/second, with several passes under the UV lamp to ensure through cure.

### EXAMPLE 3

Preparation 3

A biliquid foam was prepared from the following ingredients.

| Ingredients | % |
|---|---|
| Aqueous phase | |
| Demineralised Water | 9.9% |
| Tween 20 | 0.1% |
| Oil phase | |
| Fragrance | 89.1% |
| Castor oil/polyethylene glycol (25) additive | 0.9% |

The oil phase was added dropwise to the aqueous phase, which was stirred by a propeller impeller at 200 rpm. The product was left stirring for a further 15 minutes after the addition of the oil was complete.

Preparation of Monomer Mixture A

| | % |
|---|---|
| Craynor CN9761 | 76.35% |
| Sartomer monomer SR489 | 23.64% |

The two monomers were added together and stirred to give a homogenous mixture.

Formulation 3

| Ingredients | % |
|---|---|
| Monomer Mixture A | 74.8 |
| Preparation 3 | 21.3 |
| Darocur 1173 initiator | 3.9 |

The ingredients of the formulation were added sequentially in the order given above, with stirring to ensure a homogeneous mixture.

The formulation was then applied as a 100 micrometre thick coating to a substrate using a calibrated slot film applicator. The sample was then cured, using a GEW bench UV curing system with power level of 100 W/cm and a conveyor speed of 0.1 m/second, with several passes under the UV lamp to ensure through cure.

### EXAMPLE 4

Preparation 4

A biliquid foam was prepared from the following ingredients:

| Ingredients | % |
|---|---|
| Aqueous phase | |
| Demineralised Water | 9.9% |
| Sodium lauryl ether sulphate | 0.1% |
| Oil phase | |
| Mineral oil | 89.1% |
| Laureth 4 | 0.9% |

The oil phase was added dropwise to the aqueous phase at first. The aqueous phase was stirred using a large paddle stirrer at 110 rpm. After 5 minutes of the dropwise addition of the oil phase, the stirrer speed was maintained at 110 rpm but the oil phase was then added in a steady stream. When the addition of the oil phase was complete the product was stirred for a further 15 minutes. The product was then further sheared down at 600 rpm to decrease the droplet size.

Preparation of Monomer Mixture B

| | % |
|---|---|
| Ebecryl 2001 | 48.7 |
| Darocur 1173 | 4.5 |
| Ebecryl 2002 | 30.1 |
| Photomer 4174 | 10.6 |
| Demineralised water | 6.1 |

The monomers and water were added sequentially in the order given above, each time with stirring to give a homogeneous mixture.

Formulation 4

| | % |
|---|---|
| Monomer mixture B | 77 |
| Preparation 4 | 19.2 |
| Microflex-1(wetting agent) | 3.8 |

The ingredients of the formulation were added sequentially in the order given above with stirring to give a homogeneous mixture.

The formulation was then applied as a 100 micrometre thick coating to a substrate using a calibrated slot film applicator. The sample was then cured, using a GEW bench UV curing system with power level of 100 W/cm and a conveyor speed of 0.1 m/second, with several passes under the UV lamp to ensure through cure.

### EXAMPLE 5

Formulation 4 of Example 4 was applied to a substrate by a screen-printing process. Screen-printing was carried out using a Roku Print SD05 machine at 10% of its rated velocity, about 0.2 m/s. The screen mesh used was 180 threads per centimetre. After applying the formulation onto the substrate, curing was achieved using a GEW bench UV curing system at a power level of 100 W/cm, with several passes under the UV lamp to ensure a thorough cure.

Footnote to the Examples

| Trade Name | Chemical Name |
|---|---|
| Kathon CG | Preservative - Mixture of: 5-chloro 2-methyl- |
| | 4-isothiazolin-3-one and 2-methyl-4- |
| | isothiazolin-3-one |
| Volpo L3 | C₁₂₋₁₃ Pareth-3 |
| Volpo L4 | C₁₂₋₁₃ Pareth-4 |
| Ebecryl 2001 | aliphatic urethane diacrylate |
| Ebecryl 2002 | polyurethane acrylate/tri-propylene glycol |
| | diacrylate |
| Darocur 1173 | 2-hydroxy-2-methyl-1-phenyl-1-propanone |
| Darocur 4265 | 2-hydroxy-2-methyl-1-phenyl-1-propanone/ |
| | diphenyl(2,4,6-trimethylbenzoyl)-phosphine |
| | oxide |
| Photomer 4174 | Ethoxylated pentaerythritol triacrylate |
| Tween 20 | Polysorbate 20 |
| Craynor CN9761 | aromatic urethane acrylate |
| Sartomer monomer SR489 | triclecyl acrylate |

1. A method of coating the surface of a substrate which comprises the steps of:
i) contacting the surface with a polymerisable mixture comprising one or more polymerisable components and containing suspended droplets of a biliquid foam or of a high internal oil phase emulsion, the said droplets being stabilised by a non-reactive surfactant; and
ii) polymerising the coating to form a polymer comprising the droplets entrapped therein.

2. A method according to claim 1 wherein the coating is polymerised to form a film of the polymer comprising the droplets entrapped therein.

3. A method as claimed in claim 1 or 2 wherein a biliquid foam is used.

4. A method as claimed in claim 1 or 2 wherein a high internal oil phase emulsion is used which comprises at least 70 percent by weight of the oil phase.

5. A method as claimed in claim 4 wherein the high internal oil phase emulsion comprises at least 90 percent by weight of the oil phase.

6. A method as claimed in any one of the preceding claims wherein the polymerisable mixture comprises from 1 to 50 percent by weight of the biliquid foam or high internal oil phase emulsion.

7. A method as claimed in claim 6 wherein the polymerisable mixture comprises from 20 to 40 percent by weight of the biliquid foam or high internal oil phase emulsion.

8. A method as claimed in any one of the preceding claims wherein the coating is polymerised using electron beam, UV radiation, visible radiation, near infra-red, thermal or gamma radiation curing.

9. A method as claimed in any one of the preceding claims wherein the external phase of the biliquid foam or high internal oil phase emulsion comprises water or mixture of water with a polar solvent.

10. A method as claimed in claim 9 wherein the external phase comprises a mixture of water and a C₁₋₄ alcohol or organic oxygenate.

11. A method as claimed in any one of the preceding claims wherein the coating is polymerised by free-radical polymerisation.

12. A method as claimed in any one of the preceding claims wherein the polymerizable mixture is applied to the surface by printing.

13. A method as claimed in claim 12 wherein the printing is screen-printing, gravure printing, flexographic printing, lithographic printing, ink-jet printing or pad printing.

14. A method as claimed in any one of claims 1 to 11 wherein the polymerizable mixture is applied to the surface by spray-coating, roller coating, dip coating, or blade, pad or extrusion coating.

15. A method according to any one of claims 1 to 11 wherein the polymer comprising the droplets entrapped therein is a dental filling.

16. A method according to any one of claims 1 to 14 wherein the polymer or polymer film comprises droplets comprising a fragrance entrapped therein and is a fragranced coating.

17. A method according to any one of claims 1 to 14 wherein the surface coating is a security or tamper proof coating comprising a chemically reactive or thermo-chromic or photo-chromic dye.

18. A surface coating prepared according to any one of the preceding claims which comprises droplets of a biliquid foam or high internal oil phase emulsion entrapped within a polymer or polymer film.

19. A surface coating as claimed in claim 18 in which the polymer or polymer film is selected so that the oil phase of the biliquid foam or high internal oil phase emulsion is releasable from the coating upon the application of shear force to the polymer or polymer film.

20. A surface coating as claimed in claim 18 in which the polymer or polymer film is selected so that the oil is releasable from the coating by the action or a chemical release agent on the polymer.

21. A surface coating as claimed in claim 20 in which the oil is released at a predetermined pH.

22. A surface coating as claimed in claim 20 in which the oil is releasable by contact of the polymer film with water, or other predetermined solvent.

23. A surface coating as claimed in claim 18 in which the polymer or polymer film is selected so that the oil is releasable from the coating by the application of heat to the polymer.

24. A surface coating as claimed in any one of claims 18 to 23 in which the polymer or polymer film is partially or wholly crosslinked.

25. A stand alone polymer or polymer film which is obtained by removing the surface coating as claimed in any one of claims 18 to 24 from the substrate on which it is formed.
